(19) United States
(12) Patent Application Publication
(10) Pub. No.: US 2011/0177591 A1
(43) Pub. Date: Jul. 21, 2011

(54) METHOD AND DEVICE FOR PRODUCING DERMIS

(75) Inventors: Peter Iwatschenko, Neunkirchen (DE); Hans Gärtner, Eckental (DE); Arnd Wilhelmi, Forchheim (DE)

(73) Assignee: TUTOGEN MEDICAL GMBH, Neunkirchen am Brand (DE)

(21) Appl. No.: 12/993,493

(22) PCT Filed: May 21, 2008
(86) PCT No.: PCT/EP08/04091

§ 371 Date: Feb. 18, 2011

(51) Int. Cl. C12N 5/071; C12M 1/00

(52) U.S. Cl. 435/325; 435/283.1

(57) ABSTRACT

A process and a device for preparing a dermis implant of a predefinable thickness provides for a section of skin (30) to be laid on a plate (20). Subcutaneous material is removed with a milling cutter (40) until the skin is of the desired thickness.

### TECHNICAL FIELD

The disclosure relates to a process and a device for preparing dermis as a graft.

### BACKGROUND

A tissue or organ to be transplanted is generally referred to as a transplant or graft. The dermis is one layer of the organ which covers the body, known as the “skin” (cutis). These terms are used in connection with mammals and also in particular in connection with the human skin. Roughly speaking, the skin is generally divided into three layers, the epidermis, the dermis and the subcutis. In order to provide a suitable graft for many surgical procedures, the skin obtained from a donor, for example, has to be freed from the subcutis. The subcutis consists in particular of adipose fat, nerves, blood vessels, strands of connective tissue, etc.

The outer skin layer, or epidermis, is separated from the dermis beneath it by the stratum basale. The dermis is largely composed of connective tissue cells (fibroplasts), which form an extracellular matrix (ECM). The main components of these cells are fibrous proteins (collagens). Two layers of the dermis are normally distinguished, the reticular dermis (stratum reticulare) facing the subcutis and the papillary dermis (stratum papillare) facing the epidermis.

The prior art is familiar with the use of grafts obtained from skin, wherein both humans and suitable animals, for example pigs, make appropriate donors. Skin is taken from these tissue donors, treated by means of chemical processes, and sterilised. Such grafts are used for example in the treatment of major defects in the abdominal wall, in particular incisional hernias. The collagen structure of the implant implanted in this way acts as a guide for the growth of new tissue by the body. At the implant site the degradation of the implant and the formation of new connective tissue by the body occur in parallel, so that within a few weeks the soft tissue defect is closed by a scar. Alternatively, such indications can also be treated by the implanting of synthetic meshes, which are intended to cover the defect at the hernial orifice. The disadvantage of such synthetic meshes is their encapsulation and the additional operation that is subsequently necessary.

Prepared dermis intended for use as a graft for the treatment in particular of soft tissue defects in a patient first undergoes an inactivation process to prevent undesirable complications (infection, rejection).

The known inactivation processes include steps to inactivate microorganisms, viruses, prions and cells from the tissue donor. This ensures optimal tolerance and safety of the implant. The prior art is familiar with the process known as the Tutoplast process for inactivation as described above and preservation to prepare a ready-to-use sterile graft. The present disclosure assumes that this Tutoplast process is known and can also be used here.

The process includes in particular the following steps for the preparation of a skin graft:

Breakdown of the tissue cells using alternating baths of sodium chloride solutions and demineralised water to mobilise intracellular proteins;

Washing to remove cellular and extracellular components;

Inactivation of bacteria and viruses, etc., using sodium hydroxide solution

Inactivation of bacteria and viruses, etc., using hydrogen peroxide;

Inactivation of viruses and removal of water using a solvent (in particular acetone); and

Removal of acetone by drying.

The tissue is dehydrated during the Tutoplast process, and this leads to a non-uniform contraction.

If the doctor then wishes to use the graft prepared in this way on the patient, the dry product must first be rehydrated at the place of use using physiological sodium chloride solution and restored to its condition prior to solvent preservation.

In the Tutoplast process described above, the skin from the donor (for example the human organ donor) must first be prepared. For example, large amounts of fatty tissue (subcutis) adhere to the papillary dermis, and in the prior art these have to be removed very carefully in order not to damage the tissue structure. In particular, the poor dimensional stability of the dermis section presents difficulties here. The skin section is very difficult to hold during this process if the fatty tissue has to be removed using a scalpel.

A very experienced lab technician needs about 20 minutes to prepare a piece of skin measuring 200 mm×300 mm. The incision follows the boundary between the stratum reticulare and the fatty tissue of the subcutis (the adipose fat). The resulting thickness of the dermis graft is the same as the thickness of the donor's skin. However, the thickness of each donor's skin differs according to their age, sex and constitution. The donor-related differences in skin thickness thus result in grafts of differing thicknesses after preservation, i.e. in particular after the use of the Tutoplast process described above. In addition, the reticular side of the resulting graft is often extremely irregular.

As explained above, the dry dermis preserved using the Tutoplast process must be rehydrated before it can be used with a patient. However, the swelling behaviour that occurs during rehydration is non-uniform and dependent on the tissue, which means that it is almost impossible to predict the change in thickness of the resulting product in the wet state as compared with its original (dry) slate. This means that the thicknesses of two sections of dry dermis from different donors may be very different after rehydration.

### SUMMARY

The object of the disclosure is to provide a process and a device for preparing a dermis graft by means of which a uniform graft thickness can be made available to a user (surgeon).

To this end the disclosure teaches a process for preparing a dermis graft of a predefinable thickness, wherein

a) a section of skin is laid on a plate,
b) the section of skin is brought into contact with the plate in such a way that a mechanical removal of material is possible from the side of the skin directed away from the plate, and
c) material is removed from said side of the skin by means of a tool.

The disclosure thus comprises the mechanical cutting of the skin in the wet, i.e. raw, state to a desired thickness and the subsequent use of the Tutoplast process, for example, for preservation, as described above.

Although the tissue contracts (as described above) during the Tutoplast process, when it is rehydrated in the manner described the tissue reverts to the thickness it acquired on completion of the process according to the disclosure using the device according to the disclosure. The disclosure thus allows the user (surgeon) to be provided with a graft which after rehydration at the place of use has a precisely defined thickness, so that if several graft sections are used, the surgeon is able for example to use only sections which after rehydration, i.e. when used on the patient, are of equal thickness.

According to the disclosure the processing of a section of skin obtained from a donor (human or non-human) thus takes place when the tissue is hydrated. In this state the section of skin to be processed is attached to a plate substantially via its entire surface and then the side directed away from the plate is cut by mechanical means to remove the elements not required for the graft. The section of skin is attached to said plate via its entire surface so that said mechanical cutting can take place without distortion of the skin. A particularly elegant means of attaching the section of skin to be cut to said plate is achieved by freezing when the skin is wet. Here the plate is cooled to a temperature at which the skin freezes sufficiently solidly to the plate to allow the subsequent mechanical cutting to take place, preferably with a milling cutter, for example. In an analogy to the mechanical cutting of metallic work-pieces, for example, the preparation method according to the disclosure can also be described as “machining”. The freezing to the plate of the skin to be cut can be encouraged for example by wetting the plate and/or the papillary side of the skin to be laid on the plate with a freezing liquid, for example water.

The device according to the disclosure for preparing a dermis graft of a predefinable thickness is provided with

a plate with which a section of skin can be brought into contact, and

a tool for removing material from the side of the skin directed away from the plate.

According to a preferred embodiment the device has means for cooling said plate in such a way that a section of skin to be cut adheres sufficiently solidly to the plate and has a sufficiently rigid structure to allow mechanical cutting, with a milling cutter, for example.

A milling cutter is preferably used as the tool for mechanical cutting, but in general terms another tool having cutting means can also be used, in particular a rotary tool having cutting means.

According to a preferred embodiment of the disclosure, a heatsink on which the plate can be removably rested is used to cool down the plate. This makes it possible to remove the plate easily from the device to clean and sterilise the plate. A number of plates can then also be used in succession in a single device.

The tool used, for example the milling cutter, can also be mounted in the device in such a way that, similarly to the plate, it can be mounted in the device so as to be removed from one section of skin to the next, while tools (milling cutters) not currently in use can be cleaned and sterilised.

The versions of the disclosure described above allow the tissue to be treated to be attached very quickly to a smooth plate by freezing. With the tissue in this adequately frozen state, subdermal fatty tissue in particular can be quickly and effectively removed. In addition, irregularities on the reticular side of the dermis can be levelled out neatly and the thickness of the dermis can be reduced to exactly the required dimension. Even after subsequent drying, for example by means of the aforementioned Tutoplast process, followed by rehydration performed by the surgeon intending to use the graft, the graft has exactly the thickness it acquired after cutting according to the disclosure, and this thickness can be specified for example on the packaging of the graft, which is conventionally supplied in the dry state.

Uncertainties regarding the thickness of the graft owing to the contraction effects described above are thus avoided.

The disclosure allows a very considerable time saving in the preparation of the graft and also a most appreciable improvement with regard to the graft quality and the reproducibility of the preparation results.

An embodiment example of the disclosure is described in more detail below by reference to the drawing. The description relates to both the process and the device.

### BRIEF DESCRIPTION OF THE DRAWINGS

FIG. 1 shows a schematic view of an embodiment example of a device for preparing a dermis graft; and

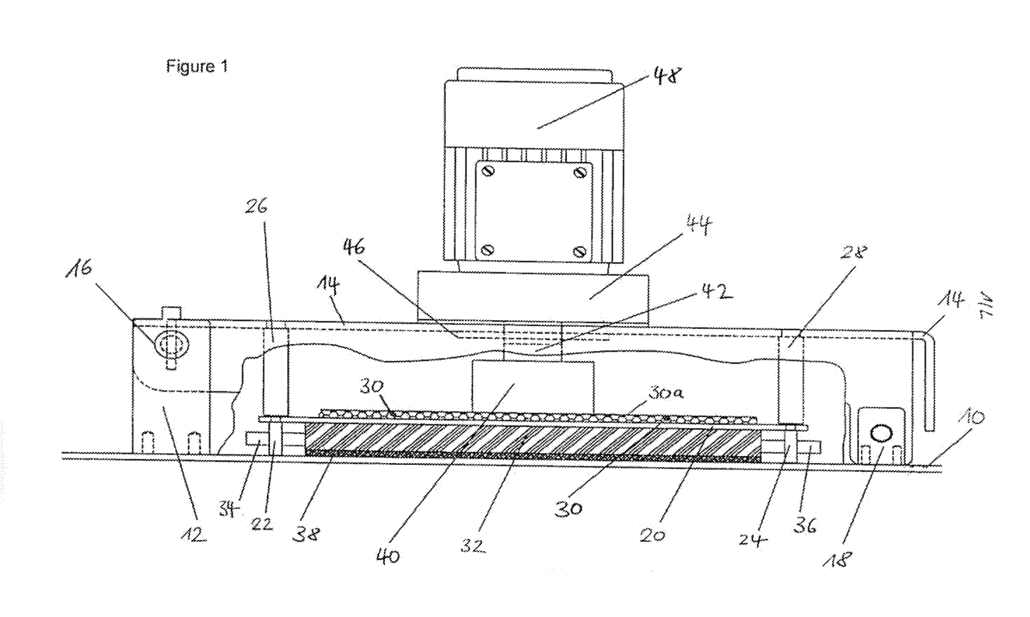

FIG. 2 shows a top view of the device as illustrated in FIG. 1.

### DETAILED DESCRIPTION

The aim of the cutting of a section of skin from a donor using the device as shown in the figure is to prepare a graft for use on a human being, said graft having a predefinable thickness when wet. For example, a section of skin as obtained from a donor as starting material can have a thickness in the range from 4 to 7 mm and on completion of the process using a device as shown in the figure the graft should then have a thickness in the range from 3 to 4 mm, for example.

The starting material, in other words the section of skin, can be supplied in a sodium chloride solution for example or in a frozen state.

The device shown in the figure has a base plate 10, which serves to support the substantial components of the device. The figure illustrates substantial components of the device from the side, some in cross-section.

An upper housing section 14 is pivot-mounted on bearing blocks 12 (for example two bearing blocks). The axis of rotation is labelled with reference numeral 16. The figure shows the state of the device with the upper housing section 14 closed. To open the device the upper housing section 14 in the figure can thus be swung open in an anticlockwise direction about the axis of rotation 16. Locking brackets 18 serve to lock the upper housing section 14 in the closed state, in which the cutting of the skin described below takes place. In this closed state the upper housing section (part of which is shown broken away in the figure) completely covers the skin to be cut and the shreds of tissue that are stripped away, so that no shreds of tissue produced during the process can escape from the device in an uncontrolled manner. The upper housing section can optionally be provided with a transparent window to enable a user to visually monitor the cutting process.

A plate 20 is supported above the base plate 10 by means of several vertical journals 22, 24 (only two illustrated). When the upper housing section 14 is closed, hold-down clamps 26, 28 press the plate 20 onto a heatsink 32 in such a way that good thermal contact is established between the heatsink 32 and the plate 20.

When the upper housing section 14 is open, the hold-down clamps 26, 28 do not act on the plate 20, so that this can be lifted away from the heatsink 32.

The section of skin 30 to be cut is laid on the plate 20 with its entire surface in contact, and any air influences or similar are smoothed out.

The skin has an upper side 30a corresponding to the subcutis, i.e. the removal of tissue (for example fat, etc.) should lake place on the upper side 30a of the skin 30.

The heatsink 32 has an inlet 34 for a cooling medium and an outlet 36 for the cooling medium. Between the heatsink 30 and the base plate 10 there is an insulating plate 38 so that no cooling action is exerted unnecessarily on the base plate 10 but rather cooling extends substantially to the plate 20. A temperature sensor (not illustrated) can be attached to the plate 20 to control the temperature of the plate 20. The temperature sensor is provided with a controller for the heatsink 32 to this end. If the heatsink 32 is cooled in the manner described above by means of the flow of a cold medium through the inlet 34 and the outlet 36, then the controller controls the temperature of the medium in the manner of a control circuit according to the measured temperature of the plate 20. In the version illustrated in the figure, liquid carbon dioxide, nitrogen, etc., are suitable in particular as cooling media.

As an alternative to the heatsink 32 described above, which is cooled by a flow of cooling medium, other cooling methods can also be used, for example solid-state cooling methods (Peltier element).

The temperature to be established on the plate 20 is in the range from 0° C. to −50° C., as required.

In the embodiment example illustrated, the device has a milling cutter 40, which is guided over the upper side 30a of the section of skin 30 in such a way that the desired removal of tissue takes place. The milling cutter 40 can therefore be guided in paths across the surface 30a of the skin 30. In one version the process can be guided by hand, wherein the user monitors the progress of the tissue removal visually (through said window) and stops the process when the visible surface of the skin has attained a desired structure. The milling cutter 40 can however also be guided and controlled mechanically in such a way that a predefinable final thickness of the skin can be entered by the user via a computer and the milling cutter then cuts the skin in accordance with a predefined, optionally multilayer removal program. To this end according to one version of the device an instrument for measuring the thickness of the skin 30 can be provided, for example an optical measuring instrument using the surface of the plate 20 as a reference point. In this way the actual thickness of the skin can be measured and compared with a target thickness to be attained at the end of the cutting process, and the computer (or user) guides the milling cutter 40 across the skin 30 until all parts of the skin are of the desired target thickness.

In order to alter the relative position of the milling cutter 40 in relation to the skin, a milling cutter journal 42 supporting the milling cutter 40 can be adjusted by means of a height adjuster 44. The milling cutter 40 is turned by means of an electric motor 48.

To operate the device described above, a section of skin 30 to be cut is first laid on a plate 20. The plate 20 may be outside the device shown in the figure at this point. Any bubbles of air or liquid trapped between the skin and plate are pressed out to the side using an appropriate scraper (not illustrated).

The heatsink 32 is brought to the desired temperature. Then the plate holding the section of skin with its entire surface in contact is placed on the heatsink 32 and the upper housing section 14 is swung into the closed position shown in the figure. The upper housing section 14 is secured in the closed position by the locking brackets 18 using bolts (not illustrated). This causes the hold-down clamps 26 to be pressed onto the plate 20 so that the entire surface of the plate 20 rests firmly on the heatsink 32 and assumes its temperature. When the section of skin 30 reaches the desired temperature, the cutting process can be started. If relatively thick layers of fatty tissue, etc., are to be removed from the skin, the height of the milling cutter can be progressively lowered in order to remove each layer.

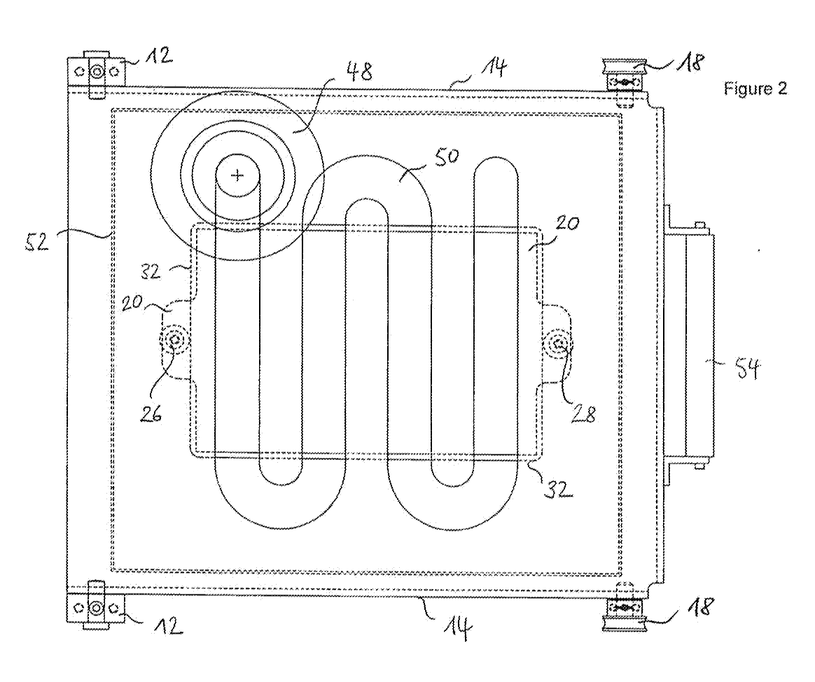

FIG. 2 shows a top view of the device described above, with the path 50 of the milling cutter traced in a serpentine pattern over the skin to be cut. A window 52 allows the operation to be visually monitored. A handle 54 serves to manipulate the device.

The device described by reference to the figure can be extended in particular with a suction device which picks up the ablated material tinder vacuum as soon as it has been removed from the skin by the milling cutter 40. A “shavings tray” can also be provided for the orderly removal of ablated material.

On completion of the process the dermis implant prepared in this way can be removed from the plate 20 and can then undergo the Tutoplast preservation described above, for example. This causes the graft to contract, but on subsequent rehydration it reverts to its original shape and thickness.

1. Process for preparing dermis of a predefinable thickness, comprising:
a) laying a section of skin onto a plate
in such a way that a mechanical removal of material is possible from a side of the skin directed away from the plate, and
b) removing material from said side of the skin by means of a tool.

2. Process according to claim 2, wherein in step a) the section of skin is substantially attached to the plate via its entire surface.

3. Process according to claim 1, wherein a connection between the section of skin and the plate is established by freezing.

4. Process according to claim 1, wherein the material is removed by means of a milling cutter.

5. Device for preparing dermis of a predefinable thickness, comprising:
a plate with which a section of skin can be brought into contact, and
a tool for removing material from a side of the skin directed away from the plate.

6. Device according to claim 5, further comprising a means to cool the plate.

7. Device according to claim 5, wherein the tool has cutting means.

8. Device according to claim 7, wherein the tool is a milling cutter.

9. Device according to claim 5 further comprising a means to cool the plate in order to bring about a full-surface adhesion of the skin to the plate.

10. Device according to claim 9, further comprising a heatsink on which the plate can be rested.

11. Device according to claim 5, wherein the plate is removably mounted in the device.

12. Device according to claim 5, wherein the tool is removably mounted in the device.
